(19) United States
(12) Patent Application Publication
(10) Pub. No.: US 2013/0098286 A1
(43) Pub. Date: Apr. 25, 2013

(54) METHOD AND AGENT FOR DETECTING DRUGS IN BEVERAGES

(71) Applicants: Catherine Herry, Saint-Ouen du Tilleul (FR); Pauline Contamin, La Feuillie (FR); Emmanuel Dupau, Rouen (FR)

(72) Inventors: Catherine Herry, Saint-Ouen du Tilleul (FR); Pauline Contamin, La Feuillie (FR); Emmanuel Dupau, Rouen (FR)

(21) Appl. No.: 13/808,807

(22) PCT Filed: Jul. 5, 2011
(86) PCT No.: PCT/FR2011/051600

§ 371 Date: Jan. 7, 2013

(30) Foreign Application Priority Data
Jul. 6, 2010 (FR) 10 55490

(51) Int. Cl. G01D 7/00; A61K 47/20; A61K 47/22

(52) U.S. Cl. CPC G01D 7/005; A61K 47/22; A61K 47/20
USPC 116/201; 424/10.3

(57) ABSTRACT

The present invention relates to a method for combating chemical submission, which comprises: putting into solution, in a beverage, a pharmaceutical form comprising an active ingredient and at least 0.05 mg, preferably from 0.2 to 5 mg, even more preferentially from 0.3 to 2 mg of at least one water-soluble colouring agent chosen from: indigocarmine or E 132, erythrosine or E 127, brilliant blue FCF, alphazurine FG, fast green FCF, quinzarine green SS, orange II, tartrazine and Sunset yellow FCF, detecting the pharmaceutical form, said detection being characterized by the immediate change in colour of the beverage; it also relates to the use of said colorant for combating chemical submission, and also to a non-film-coated solid pharmaceutical form comprising said colorant.

The subject of the invention is a method for combating surreptitious administration of chemicals.

For some years, delinquents have been using the hypnotic properties of certain substances to drug someone without their knowledge. The criminals do not hesitate to introduce surreptitiously into their victim's drink a pharmaceutical form to alter their behavior, or indeed to render them totally amnesic. Once the victim has been deprived of all awareness, the criminal can take advantage of them: theft, rape or extortion of money. Moreover, the ingestion of such a pharmaceutical form without regard to the prescribed doses can lead to serious consequences, particularly if it is absorbed with a quantity of alcohol. The pharmaceutical form can also produce harmful interactions with other medicaments which the victim could have taken beforehand.

From the state of the art, a hypnotic, Rohypnol, is known which is widely used for illicit purposes by reason of its ease of dissolution and its imperceptible nature. The formulation of this drug has been revised to result in a tablet green on the outside and blue on the inside, film-coated and thus slow to dissolve, releasing a blue color. However, the blue coloration is only visible after a quarter hour of immersion in the liquid; the victim is thus not in a position to detect the surreptitiously introduced hypnotic if they drink their drink immediately.

Also known from the state of the art is the document WO 2005/059541 which relates to a kit for detecting drugs stealthily introduced into drinks. However, this system only protects persons provided with this kit.

It is thus vital and urgent to find a method enabling immediate detection of the illicit misuse of drugs in case of surreptitious chemical administration without resorting to a detection device or kit.

An essential objective of the present invention is thus such a method utilizing a pharmaceutical form comprising at least one compound enabling immediate detection of said pharmaceutical form illicitly introduced into a drink. The invention also relates to this pharmaceutical form.

The purpose of the present invention is to offer a new method for combating surreptitious administration of chemicals. This purpose is achieved by means of a method for combating surreptitious administration of chemicals comprising:
dissolution in a drink of a solid pharmaceutical form comprising an active principle and at least 0.05 mg, preferably from 0.2 to 5 mg, still more preferably from 0.3 to 2 mg of at least one orally acceptable water-soluble coloring agent selected from the group comprising indigocarmine, erythrosine, brilliant blue FCF, alphazurine FG, fast green FCF, quinzarin green SS, orange II, tartrazine and sunset yellow FCF, and
detection of the pharmaceutical form by the immediate change in the color of the drink.

The invention relates to the utilization in a solid pharmaceutical form of at least 0.05 mg, preferably from 0.2 to 5 mg, still more preferably from 0.3 to 2 mg of at least one water-soluble coloring agent selected from indigocarmine, erythrosine, brilliant blue FCF, alphazurine FG, fast green FCF, quinzarin green SS, orange II, tartrazine and sunset yellow FCF for combating surreptitious administration of chemicals.

The invention also relates to a non-film-coated solid pharmaceutical form for combating surreptitious administration of chemicals comprising an active principle and at least 0.05 mg, preferably from 0.2 to 5 mg, still more preferably from 0.3 to 2 mg of at least one water-soluble coloring agent selected from indigocarmine, erythrosine, brilliant blue FCF, alphazurine FG, fast green FCF, quinzarin green SS, orange II, tartrazine and sunset yellow FCF, and pharmaceutically acceptable excipients.

In the present invention, “non-film-coated solid pharmaceutical form” is understood to mean any solid form which does not include a coating on its most external surface intended to be in contact with the medium wherein it is dissolved or with the mucous membranes. Thus, in the case of a tablet, the latter will not include an external coating. Nonetheless, the active substance may be present within this tablet in or in the form of coated granules.

“Surreptitious administration of chemicals” is understood to mean the administration of a psycho-active substance without the victim's knowledge for criminal or malicious purposes.

### DETAILED DESCRIPTION OF THE INVENTION

The detailed description of the various elements constituting the invention applies equally to all the subjects of the invention: method, utilization and composition.

The solid pharmaceutical form according to the invention comprises an active principle and at least one water-soluble coloring agent which enables the immediate detection of said pharmaceutical form illicitly introduced into a drink.

The coloring agent integrated into a pharmaceutical form makes it possible to color the drink into which the pharmaceutical form is introduced. This agent is particularly useful as it makes it possible to color all types of drink, whether they are clear or opaque with the exception of very dark drinks such as coffee or coca-cola.

### The Water-Soluble Coloring Agents

According to a first aspect of the invention, the water-soluble coloring agents that can be utilized in the invention are colorants soluble in any liquid at least in part comprising water and which are pharmaceutically acceptable. Such coloring agents can be selected from the following group: indigocarmine or E 132, erythrosine or E 127, brilliant blue FCF, alphazurine FG, fast green FCF, quinzarin green SS, orange II, tartrazine and sunset yellow FCF.

The coloring agent is present in a quantity sufficient to enable a coloration intense enough to be perceived with the naked eye and which can appear from the first seconds after the introduction of the pharmaceutical form into said drink. Thus, the coloring agent is present in a proportion of at least 0.05 mg, preferably from 0.2 to 5 mg, and still more preferably from 0.3 to 2 mg in the pharmaceutical form.

When the coloring agent is indigocarmine, a blue color is released immediately from the pharmaceutical form, coloring for example the drink blue if it is a colorless drink such as water or lemonade or green if it is a yellow drink such as orange juice.

Erythrosine, another coloring agent, colors the drinks red.

In the present invention, “immediate” is understood to mean the change in the organoleptic properties of the drink which takes place in less than one minute, preferably in less than 30 seconds, still more preferably in less than 15 seconds, from the introduction of the pharmaceutical form into the drink. For example, in the context of the present invention, the appearance of or change in the color can take place in less than 30 seconds, preferably in less than 15 seconds.

According to another aspect of the invention, the term “immediate” can also be defined as the change in the organoleptic properties of the drink which takes place in less than one minute, preferably in less than 30 seconds, still more preferably in less than 15 seconds from the introduction and the stirring of the pharmaceutical form into the drink.

“Stirring” is understood to mean a setting of the liquid in motion, for example by means of a straw, spoon or by movement of the vessel.

According to different particular embodiments of the invention and apart from the coloring agents which are described above and which make it possible to color a doped drink, the detection of the active substance in the drink can also be effected through the presence in the solid pharmaceutical form of at least one compound selected from the group comprising:
opacifying agents, and/or
fluorescent agents, and/or
floating particles, and/or
particles perceptible in the mouth, and/or
effervescent microgranules.

In the context of the invention, said compounds can be integrated into the solid pharmaceutical form singly or in combination. For example, a pharmaceutical form containing a coloring agent together with floating particles could be created, or indeed a solid pharmaceutical form comprising a coloring agent and a mixture of the compounds described above could be proposed.

The solid pharmaceutical form advantageously comprises at least one coloring agent with floating particles and/or particles perceptible in the mouth and/or effervescent microgranules.

### Opacifying Agents

Opacifying agents are inorganic compounds which make it possible to make drinks cloudy. These may be silicates such as magnesium silicate, aluminum silicate (in particular kaolin), magnesium aluminum silicate, calcium silicate, titanium dioxide and mixtures thereof. These compounds are generally present in a quantity of at least 15 mg, preferably from 15 to 100 mg, more preferably from 20 mg to 60 mg and still more preferably from 25 to 40 mg. Below 15 mg, the opacity could prove more difficult to detect with the naked eye.

The opacifying agents will make it possible to make drinks into which the pharmaceutical form is introduced cloudy. Thus, the drink will not only change color, but will become cloudy which will further attract the attention of the person for whom that drink is intended and will thus facilitate the detection of an undesired active substance.

These agents are particularly useful for rendering cloudy transparent and clear drinks such as water, white wine, apple juice, and spirits such as vodka, white rum, etc.

The opaque appearance of the drink appears from the first seconds after the introduction and preferably stirring of the pharmaceutical form into said drink, concomitantly with the change in color.

### Fluorescent Agents

The solid pharmaceutical form can also contain a fluorescent agent in a quantity of at least 0.1 mg, preferably in a quantity of at least 1 mg, more preferably between 0.2 and 5 mg, and still more preferably between 0.3 and 2 mg. This agent can be fluorescein and derivatives thereof, or indocyanine green.

This agent is visible in all types of drink in the presence of ultraviolet rays and in the dark. It makes it possible to reveal the pharmaceutical form containing it by emitting fluorescent light which is emitted from the doped drink. This agent is particularly useful for warning the victim when they are in a dark space where it is easy to stealthily introduce a foreign body into a drink.

Advantageously, having become fluorescent, the doped drink appears more luminous than a doped drink containing a pharmaceutical form with coloring agent but devoid of fluorescent agent, thus making it possible to alert the victim immediately.

### Floating Particles and Particles Perceptible in the Mouth

The pharmaceutical form can contain floating particles and/or particles perceptible in the mouth. These particles are microgranules comprising a blank support which is insoluble, or rendered insoluble in water or in an alcoholic solution by coating with an insoluble polymer or by coating with a lipid material.

### Microgranules

Microgranules rendered insoluble in water or in an alcoholic solution are understood to be a blank support consisting of materials soluble in water or in an alcoholic solution covered with at least one layer of materials insoluble in water or in an alcoholic solution and the function whereof is to limit or indeed to prevent the penetration of said media towards the core of the support.

The blank support insoluble in water or in an alcoholic solution advantageously comprises at least one excipient of hydrophobic nature selected from: cellulose, cellulose derivatives (microcrystalline cellulose), phosphate derivatives (calcium phosphates), silica and silicate derivatives (magnesium silicate, aluminum silicate and mixtures thereof) and carnauba wax.

In the context of the present invention, a blank support soluble in water or in an alcoholic solution can also be utilized. The soluble blank support can comprise at least one excipient selected from: starch, saccharose, polyols such as mannitol or lactose and mixtures thereof.

It is essential that this soluble blank support be rendered insoluble in water or alcohol by covering it with a coating layer either of:
polymeric nature comprising at least one hydrophobic polymer and possibly an inert filler and/or a plasticizer and/or a surfactant,
or lipid nature comprising at least one lipid material.

In the context of the present invention, the insoluble blank support can also be covered with at least one coating layer as described above, provided that this does not disadvantageously increase the density of the particles.

The coating ratio represents the ratio between the quantity of dry mass constituting the coating layer over the total mass of the microgranule before coating (as dry mass). The coating ratio lies between 0.1% to 50% m/m, preferably from 2% to 30% m/m, and still more preferably from 5% to 40% m/m.

The coating ratio is such that the particles obtained have a density less than that of the drink into which they are to be introduced, preferably a density less than 1, such that they remain on the surface of the drink into which they are to be introduced. Such particles are called floating particles.

### Polymeric Coating Layer:

The hydrophobic polymer utilized to ensure the insoluble nature of the microparticles is selected from the following group of products: non-water-soluble cellulose derivatives, (meth)acrylic (co)-polymer derivatives, polyvinyl acetate derivatives and mixtures thereof. More preferably, the hydro-phobic polymer(s) is (are) selected from the following group of products: ethylcellulose, cellulose acetate butyrate, cellulose acetate, the type A and type B ammoniomethacrylate copolymers sold under the trade name Eudragit®, in particular Eudragit® RS 30D, Eudragit® NE 30D, Eudragit® RL 30D, Eudragit® RS PO and Eudragit® RL PO of the poly(ethyl acrylate, methyl methacrylate, trimethylammonioethyl methacrylate) family, polyvinyl acetates and mixtures thereof.

The quantity of hydrophobic polymer lies between 50% to 100%, preferably from 70% to 100%, of the dry mass of the coating layer.

An inert filler can be present in the coating layer in a proportion from 0 to 50% m/m, preferably from 0 to 20% m/m and still more preferably from 5 to 20% of the dry mass of the hydrophobic coating polymer.

The inert filler uniformly distributed in the coating is selected from the group comprising in particular talc, anhydrous colloidal silica, magnesium stearate, glycerol monostearate and mixtures thereof.

When the coating is effected by an aqueous route, a plasticizer can be added to the coating dispersion in a proportion from 0% to 50% m/m, preferably from 2% to 25% m/m, in dry mass of hydrophobic coating polymer.

The plasticizer is in particular selected from the following group of products: glycerol and esters thereof, preferably from the following subgroup: medium-chain triglycerides, acetylated glycerides, glyceryl monostearate, glyceryl triacetate, glyceryl tributyrate, phthalates, preferably from the following subgroup: dibutyl phthalate, diethyl phthalate, dimethyl phthalate and dioctyl phthalate, citrates, preferably from the following subgroup: acetyl tributyl citrate, acetyl triethyl citrate, tributyl citrate and triethyl citrate, sebacates, preferably from the following subgroup: diethyl sebacate and dibutyl sebacate, adipates, azelates, benzoates, chlorobutanol, polyethylene glycols, plant oils, fumarates, preferably diethyl fumarate, malates, preferably diethyl malate, oxalates, preferably diethyl oxalate, succinates, preferably dibutyl succinate, butyrates, esters of cetyl alcohol, malonates, preferably diethyl malonate, castor oil (the latter being particularly preferred), and mixtures thereof.

More preferably, the plasticizer is selected from the following group of products: acetylated mono-glycerides, in particular Myvacete 9-45, triethyl citrate (TEC), dibutyl sebacate, triacetin, and mixtures thereof.

The surfactant is optionally present in the coating in a proportion of 0 to 30% m/m, preferably from 0 to 20% m/m, and, still more preferably, from 5 to 15% of the dry mass of plasticizer. The surfactant is preferably selected from the following group of products: alkali or alkaline earth metal salts of fatty acids, sodium dodecyl sulfate and sodium docusate being preferred, polyethoxylated oils, preferably polyethoxylated hydrogenated castor oil, polyoxyethylene-polyoxypropylene copolymers, poly-ethoxylated sorbitan esters, polyethoxylated castor oil derivatives, stearates, preferably of calcium, magnesium, aluminum or zinc, polysorbates, stearyl-fumarates, preferably of sodium, glycerol behenate, benzalkonium chloride, acetyltrimethylammonium bromide, cetyl alcohol and mixtures thereof.

### Lipid Coating Layer:

The microgranules can also be coated by coating with a lipid material.

The lipid material according to the invention is selected in particular from the following group of products: glyceryl palmitostearate, waxes, polyoxyl-glycerides and glyceryl behenate.

The quantity of lipid material lies between 50 and 100%, preferably between 80 and 100%, of the dry mass of the coating layer.

The quantity of lipid material is selected such that the density of the resulting particles is less than that of the drink into which they are to be introduced, preferably a density less than 1, such that they remain on the surface of the drink into which they are to be introduced.

The floating particles exhibit a total diameter (blank support, optionally coated if necessary) lying between 50 and 500 μm, preferably between 200 and 500 μm so as not to be perceptible in the mouth and to ensure some comfort to the patient. On the other hand, the particles perceptible in the mouth exhibit a total diameter greater than 500 μm, preferably greater than 1 mm, so as to be perceived by the lips and above all by the taste buds. The diameter of the floating particles and those perceptible in the mouth is measured by dry method laser granulometry (Malvern laser granulometer: Mastersizer 2000).

Entirely advantageously, said particles perceptible in the mouth are floating particles.

The quantity of the floating particles and/or the particles perceptible in the mouth contained in the pharmaceutical form is at least 25 mg, preferably 40 mg.

Preferably, the particles which float and/or are perceptible in the mouth can be colored by means of at least one of the following coloring agents: indigocarmine, erythrosine, brilliant blue FCF, alphazurine FG, fast green FCF, quinzarin green SS, orange II, tartrazine, sunset yellow FCF and/or can be rendered fluorescent by means of a fluorescent agent selected from the group comprising fluorescein and derivatives thereof and indocyanine green.

Advantageously, the active principle can also be colored with at least one colorant as described above so as to prevent possible sorting between the active principle and the particles which float and/or are perceptible in the mouth.

Also advantageously, the particles which float and/or are perceptible in the mouth are suitable for all types of drink.

From the introduction of the pharmaceutical form into the drink, the floating particles immediately rise to the surface of the drink and are visible to the naked eye. These particles remain on the surface of the liquid for at least 5 minutes and preferably for at least 4 hours, more preferably for at least 12 hours.

The particles perceptible in the mouth can also be floating particles. These are detected immediately by the victim on taking the first mouthful of the doped drink.

### Effervescent Microgranules

The solid pharmaceutical form can also contain effervescent microgranules. The effervescent micro-granules contain a basic excipient which will create an effervescence when it is in the presence of an acidic drink of the soda or beer type.

According to a first aspect, the microgranules comprise a blank support (soluble, insoluble or rendered insoluble) coated with particles of an alkaline agent selected from the group comprising sodium bicarbonate, calcium carbonate, or mixtures thereof.

The quantity of alkaline agent is at least greater than 5 mg, preferably greater than 10 mg and still more preferably greater than 20 mg.

When the pharmaceutical form containing the effervescent microgranules is introduced into an acidic drink, the particles of alkaline agent(s) on contact with the acid present create an effervescence visible to the naked eye.

According to a particular embodiment of the invention, the effervescent microgranules may be coated. The coating is sufficiently permeable to allow the release of particles of effervescent agent over a period of at least thirty minutes to one hour. The coating contains at least one insoluble polymer of the family of cellulose derivatives, vinyl derivatives or acrylic derivatives. It can contain a plasticizer and/or a surfactant. It can be permeabilized by addition of a soluble porogenic agent such as for example soluble derivatives of cellulose, povidone or a disintegrating agent.

The quantity of effervescent microgranules contained in the pharmaceutical form is at least 25 mg, preferably 40 mg.

Preferably, the effervescent microgranules can be colored by means of at least one coloring agent selected from indigocarmine, erythrosine, brilliant blue FCF, alphazurine FG, fast green FCF, quinzarin green SS, orange II, tartrazine, sunset yellow FCF and/or can be rendered fluorescent by means of a fluorescent agent selected from the group comprising fluorescein and derivatives thereof and indocyanine green.

Thus, effervescence will appear on the surface of the drink after introduction of the pharmaceutical form containing said microgranules.

### Active Principle

The invention is suitable for any active principle which modifies the patient's state of consciousness. More particular, the active principle is selected from the group comprising: anxiolytics for example the benzodiazepines, hypnotics, sedatives, and analgesics for example of the opioid type.

The anxiolytics are a class of psychotropic drugs, preferably selected from Alprazolam, Bromazepam, Chlordiazepoxide, Clobazam, Clonazepam, Clotiazepam, Clorazepate, Diazepam, Estazolam, Flunitrazepam, Lorazepam, Lormetazepam, Midazolam, Nitrazepam, Nordazepam, Oxazepam, Prazepam, Temazepam, Tetrazepam, Triazolam, clozapine, olanzapine, pirenzepine, zolpidem, zopiclone, zaleplon, meprobamate and etifoxine.

The opioids are preferably selected from Alfentanil, Anileridine, Butorphanol, carfentanil, Codeine, Diamorphine (heroin), Dextropropoxyphene, the Encephalins, the Endorphins, Fentanyl, Hydrocodone, Hydromorphone, Methadone, Morphine, Nalbuphine, Oxycodone, Oxymorphone, Pentazocine, Pethidine (meperidine), Propoxyphene, Remifentanil, Sufentanil, Tramadol and Buprenorphine.

According to a particular aspect of the invention, the active principle present in the pharmaceutical form is in solid form.

According to a particular embodiment, the active principle can also be colored by means of at least one coloring agent. The coloring agent can be one of those described above and/or can be rendered fluorescent by addition of a fluorescent agent such as described above.

According to another embodiment, the active principle can be coated onto the particles which float and/or are perceptible in the mouth.

### Drink

In the present application, the term drink will be used to designate cold drinks and hot drinks, for example water; sparkling water; wine (red, white or rosé); beer (brown or light); liqueurs; spirits such as vodka, rum, brandy, tequila, whisky; cocktails; fruit juices such as orange juice or grape juice; sodas such as coca-cola or lemonade; coffee; tea or herb tea. These drinks are given as an indication but in no way restrictively.

In the present invention, the vessel containing a drink into which the pharmaceutical form may be introduced has a capacity lying between 3 cl and 1 L.

### The Production Process

Depending on its nature, the active principle can be in the form of microcrystals, microgranules or brought into suspension and coated onto a blank support.

When it is coated onto a blank support, the active principle is in the form of a solution or suspension in an aqueous or organic solvent. A binder, a diluent and/or an antistatic agent can also be added.

The blank support can be any chemically and pharmaceutically inert excipient, existing in particulate, crystalline or amorphous form. By way of example, derivatives of sugars such as lactose or saccharose, hydrolyzed starch (maltodextrins) or also celluloses, are cited. Mixtures such as saccharose and starch or based on cellulose are also used for the preparation of spherical blank supports.

The active principle can also be made into the form of microgranules by a process known per se such as, for example, extrusion-spheronization, coating of the active principle in a perforated turbomixer, in a fluidized bed and others.

The various processes for production of microgranules by dry or wet granulation, presented in “Remington's pharmaceutical Sciences, 16^{th} Ed., 1980, Mack Publ. Co. of Easton, Pa., USA” can be utilized in the present invention.

The active principle can be coated with a polymer selected on the basis of the type of release desired (immediate, controlled or delayed) or its taste-masking properties.

The active principle is next combined with at least one colorant, possibly with another compound making it possible to combat surreptitious administration of chemicals, and with at least one pharmaceutically acceptable excipient.

The invention is suitable for any pharmaceutical form. According to an advantageous embodiment, the invention relates to a solid non-film-coated pharmaceutical form for combating surreptitious administration of chemicals comprising an active principle and at least 0.05 mg, preferably from 0.2 to 5 mg, still more preferably from 0.3 to 2 mg of at least one water-soluble coloring agent selected from indigocarmine, erythrosine, brilliant blue FCF, alphazurine FG, fast green FCF, quinzarin green SS, orange II, tartrazine and sunset yellow FCF enabling the immediate modification of the color of a drink into which said solid non-film-coated pharmaceutical form is introduced.

Such a pharmaceutical form can in particular be an oral form selected from non-coated tablets such as conventional tablets, suckable tablets, sublingual tablets, chewable tablets, effervescent tablets, dispersible tablets, orodispersible tablets, a powder for sachets or gel capsules, and thin films.

The invention is more especially useful for immediate release pharmaceutical compositions, since the criminal will want the effect of loss of vigilance to be as rapid as possible. It could however be adapted to controlled release forms.

Those skilled in the art know how to adapt the formulation depending on the pharmaceutical form and the desired release.

The pharmaceutically acceptable excipients utilized in the solid non-film-coated pharmaceutical compositions according to the invention are conventionally used excipients.

The following may for example be cited:
binders: for example cellulose derivatives such as HPMC, in particular the grades Pharmacoat® 603 and Pharmacoat® 606, or hydroxypropylcellulose or hydroxyethylcellulose, microcrystalline cellulose, polyvinylpyrrolidone derivatives, in particular the PVP K 30 grade, polyethylene glycol derivatives, in particular polyethylene glycol the molecular weight whereof lies between 600 and 7000, such as PEG4000 and PEG6000 in particular, and mixtures thereof, and vinyl derivatives such as polyvinyl alcohol;
diluents: for example soluble diluents such as lactose or mannitol, and cellulose derivatives such as microcrystalline cellulose;
preservatives: for example parabens, and antioxidants such as ascorbic acid;
solubilizers: for example poloxamers and cyclodextrins;
disintegrants: for example crospovidone and croscarmellose sodium;
sweeteners: such as aspartame and acesulfame potassium;
lubricants: magnesium stearate, sodium stearylfumarate and cotton oil;
flavors: such as mint, lemon, black cherry flavor, etc.;
surfactants: alkali or alkaline earth metal salts of fatty acids, sodium dodecyl sulfate and sodium docusate, polyethoxylated oils, preferably polyethoxylated hydrogenated castor oil, polyoxy-ethylene-polyoxypropylene copolymers, polyethoxylated sorbitan esters, polyethoxylated castor oil derivatives, stearates, preferably of calcium, magnesium, aluminum or zinc, polysorbates, stearyl-fumarates, preferably sodium, glycerol behenate, benzalkonium chloride, acetyltrimethyl ammonium bromide, cetyl alcohol and mixtures thereof; and
glidants: for example silica, talc and mixtures thereof.

In the context of the present invention, orodispers-ible tablet is understood to mean a “multiparticulate tablet disintegrating in the mouth on contact with the saliva in less than 40 seconds”. According to a particular embodiment, the invention relates to such a tablet which is based on a mixture of excipients and particles of coated active principle exhibiting intrinsic tableting characteristics. The mixing proportion of excipients relative to the particles of coated active principle is from 0.4 to 6, preferably from 1 to 4 parts by weight. The mixture of excipients comprises:
a disintegration agent or disintegrant,
a soluble diluent with binding properties,
a lubricant,
possibly, a permeabilizing agent, sweeteners and flavors,
a coloring agent making it possible to combat surreptitious administration of chemicals,
and possibly at least one of the compounds making it possible to combat surreptitious administration of chemicals, selected from opacifying agents, fluorescent agents, floating particles, particles perceptible in the mouth, and/or effervescent microgranules.

The proportion of disintegration agent and of soluble agent relative to the mass of the tablet being from 1 to 15%, preferably from 2 to 7% by weight for the first and from 30 to 90%, preferably from 40 to 70% by weight for the second.

The soluble diluent with binding properties consists of a polyol with fewer than 13 carbon atoms taking either the form of the directly tabletable product the mean diameter of the particles whereof lies between 100 and 500 micrometers, or in the form of a powder the mean diameter of the particles whereof is less than 100 micrometers, this polyol preferably being selected from the group comprising mannitol, xylitol, sorbitol and maltitol, the sorbitol not being usable alone.

The disintegration agent is selected from the group comprising in particular the crosslinked sodium carboxymethylcellulose known in the art by the term croscarmellose, crospovidone and mixtures thereof. Through the choice and the proportion of this disintegration agent, the tablet retains an acceptable hardness for normal tablets handling conditions when they are kept in sealed packaging up to temperatures of at least 30° C.

The lubricant preferably utilized in this mixture of excipients is selected from the group comprising magnesium stearate, sodium stearylfumarate, stearic acid, micronized polyoxyethylene glycol (micronized Macrogol 6000) and mixtures thereof. It can be utilized in a proportion of 0.05 to 2% relative to the total mass of the tablet.

As permeabilizing agent, a compound selected from the group comprising in particular silicas having a high affinity for aqueous solvents, such as the precipitated silica better known under the brand name Syloid, maltodextrins, 1-cyclodextrins and mixtures thereof is used.

The permeabilizing agent enables the creation of a hydrophilic network which facilitates the penetration of the saliva and thus contributes to better disintegration of the tablet.

The various compounds and production processes for orodispersible tablets described in FR2785538, WO0027357, FR2679451, WO 93/01805, FR2766089, WO 00/51568, FR2790387, WO 03/039520 and FR2831820 can be utilized in the present invention.

The invention will be described in more detail below, in particular by means of examples which are given solely by way of illustration.

### EXAMPLES

### Example 1

Orodispersible tablets containing 5 mg of zolpidem and having the following composition are prepared:

| Constituents | % | mg/unit |
|---|---|---|
| Zolpidem grains | 32.8 | 41.05 |
| Microcrystalline cellulose | 10.0 | 12.50 |
| Mannitol | 43.7 | 54.57 |
| Crospovidone | 10.0 | 12.50 |
| Colorant E132 | 0.4 | 0.50 |
| Aspartame | 1.0 | 1.25 |
| Flavor | 0.1 | 0.13 |
| Silica | 1.0 | 1.25 |
| Mg stearate | 1.0 | 1.25 |
| Total | 100.0 | 125.0 |

The orodispersible tablets are prepared as follows.

Firstly the zolpidem grains which have the following percentage composition are prepared:

| | |
|---|---|
| NPTAB 190 (180-220 μm) | 56 |
| Zolpidem tartrate | 13 |
| Hypromellose 603 | 7 |
| 1N HCl | 2 |
| Aquacoat ECD30 | 13 |
| Hypromellose 603 | 6 |
| Triethyl citrate | 3 |

The zolipidem tartrate is dissolved in water with the aid of HCl, then a dispersion is prepared by addition of hypromellose 603. NPTAB 190 sugar spheres and the dispersion prepared above are introduced into a GPCG1 fluidized bed (Glatt). An aqueous dispersion of aquacoat ECD30, triethyl citrate and hypromellose 603 is then introduced to obtain a taste-masking coating.

The zolpidem grains are then mixed with the tableting excipients. The powdery mixture is then tableted on a rotary tablet press (SVIAC PR12) equipped with round, convex punches, at a compression force of 5 kN.

125 mg tablets of 7 mm diameter are obtained which have the following properties:
Hardness 24N
Disintegration (measured according to monograph 2.9.1 of European Pharmacopeia 6.1): 15 secs
Friability (measured according to monograph 2.9.7 of European Pharmacopeia 6.1): 0.030.

The tablets exhibit a pleasant mouth feel.

One tablet is introduced into a transparent vessel containing 250 ml of water. An intense blue coloration appears as soon as the tablet is disintegrated.

A second tablet is introduced into a transparent vessel containing 250 ml of orange juice. The orange color of the juice immediately changes to an intense greenish color.

### Example 2

A conventional immediate release tablet containing 10 mg of Zolpidem is prepared.

| | % | Mg/unit |
|---|---|---|
| Zolpidem grains | 32.8 | 82.0 |
| Microcrystalline cellulose | 10.0 | 25.0 |
| Lactose | 32.7 | 81.75 |
| Calcium silicate | 20.0 | 50.0 |
| Povidone | 3.0 | 7.5 |
| Silica | 0.9 | 2.25 |
| Colorant E132 | 0.1 | 0.25 |
| Mg stearate | 0.5 | 1.25 |
| Total | 100.0 | 250.0 |

Firstly the zolpidem grains are prepared. The zolpidem grains are prepared as in example 1.

The Zolpidem grains are then mixed with the excipients mentioned in the above table. The powdery mixture is then tableted.

One tablet is then dissolved in a glass of pulp-free orange juice. Immediately after introduction and stirring of the pharmaceutical form, a greenish coloration and a cloudiness appear in a manner visible to the naked eye.

### Example 3

Two types of orodispersible tablet each containing 10 mg of Zolpidem and having the following formula are prepared:

| Constituents | % | mg/unit |
|---|---|---|
| Zolpidem grains | 32.8 | 82.1 |
| Microcrystalline cellulose | 9.6 | 23.90 |
| Mannitol | 30.0 | 75.00 |
| Crospovidone | 5.0 | 12.50 |
| Floating particles | 20.0 | 50.00 |
| Colorant E132 | 1.0 | 2.50 |
| Flavor | 0.1 | 0.25 |
| Silica | 1.0 | 2.50 |
| Mg stearate | 0.5 | 1.25 |
| Total | 100.0 | 250.0 |

These tablets are prepared as in example 1, utilizing the constituents in the above table.

For the first series of tablets (C1), the floating particles are prepared as follows:

NPTAB 190 (180-220 μm) blanks are coated with an aqueous dispersion of ethylcellulose, triacetin and talc. The coating factor is 30% of dry mass and the talc/polymer ratio is 1:2.

For the second series of tablets (C2), the floating particles are phosphate particles of dibasic calcium phosphate dihydrate coated with glyceryl palmitostearate. The glyceryl palmitostearate/calcium phosphate ratio is 1:4.

The tablets of both series disintegrate in less than 30 secs, and exhibit a pleasant mouth feel.

One tablet of each type is introduced into a glass of water. The disintegration takes place immediately and the water turns an intense blue color and the presence of particles on the surface is detectable with the naked eye. These floating particles are visible on the surface for more than 3 hours.

### Example 4

An orodispersible tablet containing 10 mg of Zolpidem, floating particles and a coloring agent, and having the following formula is prepared:

| | mg/unit | % |
|---|---|---|
| Zolpidem tartrate coated grains* | 80.00 | 17.8 |
| Avicel PH 200 | 45.00 | 10.0 |
| Mannitol SD 200 | 65.05 | 14.5 |
| Floating particles | 200.00 | 44.4 |
| Kollidon CL | 45.00 | 10.0 |
| Black cherry flavor | 0.45 | 0.10 |
| Aspartame | 4.50 | 1.00 |
| Sunset Yellow (E110) | 1.00 | 0.22 |
| Syloid 244 FP | 4.50 | 1.00 |
| Mg stearate | 4.50 | 1.00 |
| Total | 450.0 | 100.0 |

These tablets are prepared as in example 1, utilizing the constituents in the above table.

The floating particles are prepared as follows: NPTAB 190 (180-220 μm) blanks are coated with an aqueous dispersion of ethylcellulose and myvacet 9-45. The coating factor is 30% of dry mass and the plasticizer/polymer ratio is 24%.

After introduction into a glass of water and stirring, the form colors the medium orange-yellow and releases floating particles visible on the surface for more than three hours.

### Example 5

A conventional tablet containing floating particles based on carnauba wax microgranules and a coloring agent is prepared.

| | mg/unit | % |
|---|---|---|
| Granulated oxycodone HCl | 10.89 | 4.36 |
| Avicel PH 102 | 25.00 | 10.00 |
| Mannitol SD 200 | 133.11 | 53.24 |
| Carnauba wax pellets | 50.00 | 20.0 |
| Starch 1500 | 25.00 | 10.00 |
| Indigocarmine E132 | 1.00 | 0.40 |
| Syloid 244 FP | 2.50 | 1.00 |
| Mg stearate | 2.50 | 1.00 |
| Total | 250.0 | 100.0 |

The oxycodone is granulated with 4.1% of HPMC 603 in a high-shear mixer granulator. The active substance is then mixed with the tableting excipients of the above formula. The mixture is then tableted on a rotary tablet press (SVIAC PR12) equipped with round, convex punches, at a compression force of 16 kN.

250 mg tablets of 8.5 mm diameter are obtained which have the following properties:
Hardness 95N
Disintegration (measured according to monograph 2.9.1 of European Pharmacopeia 6.1): 3 mins
Friability (measured according to monograph 2.9.7 of European Pharmacopeia 6.1): 0.1%.

The tablets obtained after introduction into a drink immediately develop an uniform blue coloration, and release floating particles visible on the surface for more than 3 hours.

### Example 6

An orodispersible tablet containing 5 mg of anhydrous oxycodone HCl and a coloring agent is prepared.

| | % | mg/unit |
|---|---|---|
| Oxycodone HCl grains* | 22.0 | 29.65 |
| Avicel PH 102 | 10.0 | 13.50 |
| Mannitol SD 200 | 53.4 | 72.09 |
| Crospovidone | 10.0 | 13.50 |
| Indigocarmine E132 | 0.4 | 0.54 |
| Aspartame | 2.0 | 2.70 |
| Flavor | 0.50 | 0.675 |
| Syloid 244 FP | 0.50 | 0.675 |
| Mg stearate | 1.25 | 1.69 |
| Total | 100.0 | 135.00 |

The orodispersible tablets are prepared as follows.

Firstly the oxycodone grains which have the following percentage composition are prepared:

| Oxycodone HCl grains | % | mg/unit |
|---|---|---|
| NPTAB 250 | 54.0 | 16.01 |
| Oxycodone HCl | 18.3 | 5.43 |
| Hypromellose 603 | 7.6 | 2.25 |
| Eudragit NE30D vs | 16.7 | 4.95 |
| Syloid 244FP | 3.4 | 1.01 |
| Total | 100.0 | 29.65 |

The oxycodone is dissolved in water, then a dispersion is prepared by addition of hypromellose 603. Sugar spheres NPTAB 250 are introduced into a GPCG1 (Glatt) fluidized bed, and the dispersion prepared above is sprayed onto these. An aqueous dispersion of Eudragit NE30D and Syloid is then sprayed so as to obtain a taste-masking coating.

The oxycodone grains are then mixed with the tableting excipients. The powdery mixture is then tableted on a rotary tablet press (SVIAC PR12) equipped with round, convex punches, of diameter 7 mm. 135 mg tablets are obtained. From the introduction of one tablet into a glass of water followed by stirring, an intense and uniform blue coloration develops.

### Example 7

A conventional tablet containing 10 mg of oxycodone HCl and a coloring agent is prepared.

| | mg/unit | % |
|---|---|---|
| Granulated oxycodone HCl | 10.89 | 5.45 |
| Avicel PH 102 | 20.00 | 10.00 |
| Mannitol SD 200 | 144.12 | 72.06 |
| Starch 1500 | 20.00 | 10.00 |
| Indigocarmine E132 | 1.0 | 0.50 |
| Syloid 244 FP | 2.0 | 1.00 |
| Mg stearate | 2.0 | 1.00 |
| Total | 200.0 | 100.0 |

The granulated oxycodone is prepared as in example 5. It is then mixed with the tableting excipients so as to obtain 200 mg tablets of 8 mm diameter on a rotary press. This tablet dissolved in a glass of apple juice develops a visible greenish coloration in less than one minute.

### Example 8

A conventional tablet containing 10 mg of Zolpidem, a coloring agent and an opacifying agent is prepared.

| | mg/unit | % |
|---|---|---|
| Zolpidem tartrate | 10.00 | 4.0 |
| Avicel PH 200 | 25.00 | 10.0 |
| Lactose DCL 21 | 152.75 | 61.1 |
| Calcium silicate (FM 1000) | 50.00 | 20.0 |
| PVP K30 | 7.50 | 3.0 |
| Colorant E132 | 1.00 | 0.4 |
| Syloid 244 FP | 2.50 | 1.0 |
| Mg stearate | 1.25 | 0.5 |
| Total | 250.0 | 100.0 |

In this example, the active substance is mixed directly in the powder state with the tableting excipients. The mixing makes it possible to obtain, on a rotary press equipped with round punches of 8.5 mm diameter, 250 mg tablets. After introduction and stirring of one tablet into a glass of water, a blue coloration and cloudiness visible to the naked eye appear in less than one minute.

### Example 9

### Grains Coated with Zolpidem

The zolpidem tartrate is dissolved in water with the aid of HC1, then a dispersion is prepared by addition of hypromellose 603.

The Zolpidem grains are obtained by spraying the dispersion onto the NPTAB190 sugar spheres within a fluidized bed.

| | % | Mg/unit |
|---|---|---|
| NPTAB 190 (180-220 μm) | 73.19 | 22.52 |
| Zolpidem tartrate | 16.25 | 5.00 |
| Hypromellose 603 | 8.93 | 2.75 |
| 1N HCl | 1.64 | 0.50 |
| Total | 100.0 | 30.77 |

Coloration of the Zolpidem-Coated Grains.

A dispersion of Aquacoat is next prepared with HPMC, TEC (triethyl citrate) and the colorant; it is sprayed onto the active substance-coated grains within a fluidized bed.

| | % coated grains | mg per unit |
|---|---|---|
| Zolpidem coated grains | 75.97 | 30.77 |
| Aquacoat ECD30 | 10.17 | 4.12 |
| HPMC 603 | 10.17 | 4.12 |
| TEC | 2.44 | 0.99 |
| Colorant E132 | 1.24 | 0.50 |
| Total Utilized | 100.00 | 40.51 |

Coloration of the Floating Particles.

The colored floating particles are prepared as follows: NPTAB 250 blanks are coated with an aqueous dispersion of ethylcellulose and Myvacet® 9-45 containing the dissolved colorant by spraying in a fluidized bed.

| | % coated | mg/gel |
|---|---|---|
| | grains | capsule |
| NPTAB 250 (200-300 μm) | 79.33 | 79.33 |
| Aquacoat ECD 30D | 15.87 | 15.87 |
| Myvacet ® 9-45 | 3.81 | 3.81 |
| Indigocarmine E132 | 1.00 | 1.00 |
| Total Utilized | 100.00 | 100.00 |

The two populations are mixed in the proportions: 40.51 mg of zolpidem colored particles and 100 mg of colored floating particles. The two populations are not distinguishable. When the gel capsule is opened and its contents introduced into a glass of water, the blue coloration and the floating particles appear at once.

1. A method for combating surreptitious administration to a person of chemicals comprising:
dissolution in a drink of a solid pharmaceutical form comprising an active principle and at least 0.05 mg of at least one water-soluble coloring agent selected from indigocarmine, erythrosine, brilliant blue FCF, alphazurine FG, fast green FCF, quinzarin green SS, orange II, tartrazine and sunset yellow FCF, and
detection of the pharmaceutical form characterized by the immediate change in the color of the drink.

2. The method for combating surreptitious administration of chemicals as claimed in claim 1, wherein the active principle is selected from the group consisting of: anxiolytics, hypnotics, sedatives and analgesics.

3. The method for combating surreptitious administration of chemicals as claimed in claim 1, wherein the pharmaceutical form further contains an opacifying agent selected from the group consisting of silicates, titanium dioxide, and mixtures thereof.

4. The method for combating surreptitious administration of chemicals as claimed in claim 3, wherein the pharmaceutical form contains at least 15 mg of opacifying agent.

5. The method for combating surreptitious administration of chemicals as claimed in claim 1, wherein the pharmaceutical form contains a fluorescent agent selected from the group consisting of fluorescein or derivatives thereof, indocyanine green and mixtures thereof.

6. The method for combating surreptitious administration of chemicals as claimed in claim 5, wherein the pharmaceutical form contains at least 0.1 mg of fluorescent agent.

7. The method for combating surreptitious administration of chemicals as claimed in claim 1, wherein the pharmaceutical form contains floating particles and/or particles perceptible in the mouth.

8. The method for combating surreptitious administration of chemicals as claimed in claim 7, wherein the floating particles and/or particles perceptible in the mouth are microgranules which are insoluble or rendered insoluble by coating with a lipid material; or by coating with an insoluble polymer.

9. The method for combating surreptitious administration of chemicals as claimed in claim 7, wherein the particles have a diameter of 50 to 500 μm.

10. The method for combating surreptitious administration of chemicals as claimed in claim 7, wherein the pharmaceutical form contains at least 25 mg of particles that float and/or are perceptible in the mouth.

11. The method for combating surreptitious administration of chemicals as claimed in claim 1, wherein the pharmaceutical form contains effervescent microgranules.

12. A method for combating surreptitious administration to a person of chemicals comprising mixing into a drink a pharmaceutical form of at least 0.05 mg of at least one water-soluble coloring agent selected from indigocarmine, erythrosine, brilliant blue FCF, alphazurine FG, fast green FCF, quinzarin green SS, orange II, tartrazine and sunset yellow FCF.

13. A non-film-coated solid pharmaceutical form comprising an active principle, at least 0.05 mg of at least one water-soluble coloring agent selected from the group consisting of: indigocarmine, erythrosine, brilliant blue FCF, alphazurine FG, fast green FCF, quinzarin green SS, orange II, tartrazine and sunset yellow FCF, and at least one pharmaceutically acceptable excipient.

14. The pharmaceutical form as claimed in claim 13, in the form of a conventional tablet, suckable tablet, sublingual tablet, chewable tablet, effervescent tablet, dispersible tablet, or orodispersible tablet; powder for sachets or gel capsules, or thin film.

15. The pharmaceutical form as claimed in claim 14, wherein it is an orodispersible tablet comprising at least a coated active principle, at least one coloring agent selected from indigocarmine, erythrosine, brilliant blue FCF, alphazurine FG, fast green FCF, quinzarin green SS, orange II, tartrazine and sunset yellow FCF, and a mixture of excipients comprising:
a disintegrating agent
a soluble diluent with binding properties,
a lubricant, and
optionally, a permeabilizing agent, sweeteners and flavors,
and optionally at least one of the compounds making it possible to combat the surreptitious administration of chemicals selected from the group consisting of opacifying agents, fluorescent agents, floating particles, particles perceptible in the mouth, effervescent microgranules, and mixtures thereof.

16. The method for combating surreptitious administration of chemicals to a person as claimed in claim 1, wherein the quantity of the water-soluble coloring agent is from 0.2 to 5 mg.

17. The method for combating surreptitious administration of chemicals to a person as claimed in claim 3, wherein the silicates are selected from the group consisting of: magnesium, aluminum, calcium and magnesium aluminum silicate.

18. The method for combating surreptitious administration of chemicals as claimed in claim 4, wherein the pharmaceutical form contains from 20 to 60 mg of opacifying agent.

19. The method for combating surreptitious administration of chemicals as claimed in claim 6, wherein the pharmaceutical form contains between 0.2 and 5 mg of a fluorescent agent.

20. The method according to claim 12, wherein the quantity of the water-soluble coloring agent is from 0.2 to 5 mg.

21. The pharmaceutical form as claimed in claim 13, containing from 0.2 to 5 mg of a water-soluble coloring agent.
